(19) United States
(12) Patent Application Publication
(10) Pub. No.: US 2008/0139513 A1
(43) Pub. Date: Jun. 12, 2008

(54) TRANSDERMAL DELIVER OF ACTIVE AGENTS

(75) Inventors: Robert Lee, Boyertown, PA (US); Dinesh Shenoy, Germentown, MD (US); Steve Bandak, Edwards, CO (US)

Correspondence Address: FOLEY AND LARDNER LLP;SUITE 500, 3000 K STREET NW, WASHINGTON, DC, 20007, US

(73) Assignee: Novavax, Inc.

(21) Appl. No.: 11/862,150

(22) Filed: Sep. 26, 2007

Related U.S. Application Data
(60) Provisional application No. 60/847,092, filed on Sep. 26, 2006.

(51) Int. Cl. A61K 31/663; A61K 31/4535; A61K 31/485

(52) U.S. Cl. 514/108; 514/324; 514/282

(57) ABSTRACT

A composition for transdermal delivery of a non-traditional active agent is described. The compositions comprise: (a) a non-traditional transdermal active agent; (b) a solvent; (c) a non-miscible liquid; (d) a stabilizer; and (e) water, wherein the composition is formulated into an emulsion.

### CROSS-REFERENCE TO RELATED APPLICATIONS

This application claims priority to Provisional Application No. 60/847,092, filed on Sep. 26, 2006. In addition, the following application is incorporated by reference: U.S. application Ser. No. 11/714,274, filed on Mar. 6, 2007, for “Nano-structured Pharmaceutical Compositions and Methods of Making Them.”

### FIELD OF THE INVENTION

Generally, the present invention is directed to compositions for transdermal delivery of active pharmaceutical agents and methods of using the compositions. In particular, the active pharmaceutical agent may be a drug that is difficult to administer using conventional transdermal technology. Examples of such drugs include, but are not limited to, raloxifene, alendronate, and naltrexone.

### BACKGROUND

### A. Transdermal Drug Delivery

Transdermal drug delivery permits controlled release of a drug into a patient without directly invading the patient's body. This painless clinical technique can conveniently and effectively deliver drug doses into and through the patient's skin in a passive and continuous manner over the course of hours, days, or weeks. Typically, a transdermal patch can be placed essentially anywhere on the skin, such as under clothing, and is therefore discreet and cosmetically elegant. Its ease of use also increases patient compliance with drug administration. For example, an individual does not have to adhere to a strict oral regimen, perform routine injections or travel to a clinic for such treatment. Also, by delivering a drug directly into the blood stream, only a minimum effective amount of a drug is required, which can help reduce potential side effects. Furthermore, by delivering a drug directly into the skin and bloodstream, a transdermally-delivered drug bypasses the gastrointestinal tract, thereby eliminating first-pass liver metabolism, which may reduce or destroy a drug's bioactivity.

Transdermal delivery also creates steady levels of a drug in the bloodstream and helps to improve drug efficacy. Depending on various ingredients that are used to formulate the drug, as well as technical aspects of the patch, such as its design and adhesive qualities, the rate of release of the drug can be precisely manipulated. Accordingly, by applying different types of adhesive patches to the skin, more, less, or the same amount of drug is administered to an individual over a recommended course of time. Because of these advantages, a transdermally formulated drug is often perceived as more desirable than traditional drug delivery systems, such as injections and orally-administered tablets. Indeed, the drug industry has created transdermal patches for delivery of fentanyl, nitroglycerin, estradiol, ethinyl estradiol, norethindrone acetate, testosterone, clonidine, nicotine, lidocaine, prilocaine, oxybutynin, and scopolamine, as well as contraceptive patches containing ethinyl estradiol and norelgestromin. The U.S. transdermal market approached $1.2 billion in 2001.

The terms “transdermal” and “patch” imply a limited type of mechanism for delivery of a drug into a patient's body. In reality, the landscape concerning the types of transdermal devices useful for transdermal delivery is diverse. There exists, for instance, various patch designs that include, for example, drug-in-adhesive patches, multi-layer-drug-in-adhesive patches, microstructured systems, reservoir dispenser systems, membranes, penetration enhancer technologies, hydrogels, gels, micro-emulsions, and film-forming polymers.

Even though transdermal dosage forms are desirable in terms of patient compliance and other factors, there exists in the art problems with formulating drugs into transdermal dosage forms. For example, at present it is not possible to formulate all drugs, biological compounds, and therapeutic proteins for transdermal delivery. The solubility, physiochemical characteristics, and bioavailability of a drug can greatly influence its ability to be formulated into an appropriate transdermal composition.

Moreover, even if a drug can be formulated into a transdermal dosage form, the skin itself is often a barrier, limiting the number and types of drugs that can passively diffuse from the transdermal device and across the skin. This does not mean that transdermal dosage forms are not adaptable. Indeed, it is possible to forcefully drive drugs across the skin barrier, as opposed to relying on passive diffusion. For instance, techniques that help increase skin permeation include iontophoresis, which uses low voltage electrical current to drive charged drug particles across the skin, and sonophoresis, which uses low frequency ultrasonic energy for the same purpose. E-trans® technology developed by Johnson and Johnson's Alza Corporation was developed for the delivery of Fentanyl using low-level electrical current.¹ Another technique utilizes rapid burst of thermal energy to transport drugs, on the order of hundreds of daltons in size, across the skin barrier.¹ One example of such technology is the PassPort™ delivery system from Altea Therapeutics, which uses a hand-held battery device, with a patch to painlessly use thermal energy for the delivery of opioids, insulin, and vaccines.¹ Another relatively new technique comprises the use of microstructured arrays of needles, e.g., microneedles, that painlessly create micropores in the skin without bleeding when the patch is applied. The size of the newly-created pores can typically accommodate drugs that cannot be suitably prepared for the more traditional transdermal techniques. Alza Corporation's Macroflux® technology is one example.¹

Even with the availability of different devices, a drug may have to be reformulated to increase its suitability for transdermal delivery, regardless of which device appears to be the most effective. Indeed, ease of active pharmaceutical agent delivery is a key issue that faces all pharmaceutical companies that develop and commercialize therapeutic products for transdermal, as well as conventional, administration. An active pharmaceutical ingredient (API) that is readily soluble in water, for example, is not difficult to formulate into a suitable dosage form. However, formulating a poorly water-soluble API into suitable dosage forms poses a significant challenge. This is because the human body is a water based system; thus, as a condition of producing therapeutic activity, a drug must dissolve following administration. Raloxifene and alendronate are examples of drugs that are poorly water-soluble and are not readily adapted to transdermal delivery applications.

### B. Raloxifene Background

Raloxifene has a molecular weight of 473.584 g/mol, a melting point of 143-147° C., H₂O solubility of 0.25 mg/L, a high log P (log P of 5.749), and an absolute BA of 2%. Raloxifene is a solid at room temperature.

Postmenopausal women are more likely to develop osteoporosis, a condition characterized by weakened bones that fracture easily. If the peak bone mass before menopause is less than ideal, the bone loss during natural or surgically induced menopause may result in osteoporosis. Research suggests that about half of all women over the age of 60 years will have at least one fracture due to osteoporosis.

Medical treatments for osteoporosis include: hormone therapy (HT), bisphosphonates, selective estrogen receptor modulators (SERMs), parathyroid hormone, Vitamin D derivatives, and calcium supplements. The female sex hormone estrogen plays an important role in maintaining bone strength by inhibiting bone resorption. The drop in estrogen levels that occurs at menopause or following oopherectomy results in accelerated bone loss. It is estimated that the average woman loses up to 10 percent of her bone mass in the first five years of menopause.

Estrogen replacement therapy (ERT) has been shown to reduce bone loss, increase bone density in both the spine and hip, and reduce the risk of hip and spinal fractures in postmenopausal women. However, when estrogen is taken alone, it can increase a woman's risk of endometrial cancer. This risk is reduced by the administration progestin in combination with estrogen (hormone replacement therapy or HRT), for those women who have not had a hysterectomy. However, long term administration of estrogen is also associated with an increased risk of breast cancer, which may be further increased by concurrent progestin use.² Estrogens are also associated with an increased incidence of venous thromboembolism (VTE).³

A more recently developed alternative to estrogen replacement is provided by SERMs, which, by definition, exert estrogen agonist activity in target tissues while acting as estrogen antagonists in others. The rationale for the development of SERMs was to retain the beneficial effects of estrogen, including those on the skeleton in postmenopausal women, but to avoid adverse effects of estrogen on the uterus and breast. Raloxifene (Evista™, Lilly) was the first SERM approved in the United States for the treatment and prevention of osteoporosis in postmenopausal women. Raloxifene (Formula I) and raloxifene hydrochloride (Formula II) are SERMs that belong to the benzothiophene class of compounds.

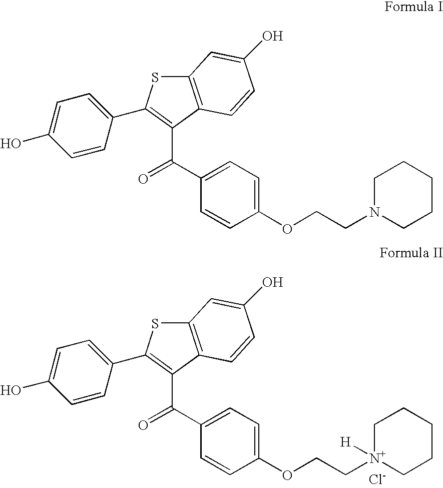

Clinical studies have indicated that raloxifene does not have uterotrophic activity, suggesting that its use may not be associated with increased risk for uterine cancer. The Multiple Outcomes of Raloxifene Evaluation⁴ (MORE) trial indicated a lower incidence of breast cancer in women taking the drug as compared with controls. An ongoing trial in more than 19,000 women over the age of 35 (the STAR trial) is comparing the efficacy of tamoxifen and raloxifene in reducing the risk of developing breast cancer. Interim analysis of the data indicates that raloxifene is as effective as tamoxifen in reducing risk for invasive breast cancer in postmenopausal women by approximately 50 percent. Women in the STAR trial, randomized to raloxifene, also had 36 percent fewer uterine cancers than did those assigned to tamoxifen.

Despite it's beneficial effects, raloxifene, like orally administered estrogen, is associated with a 2-4 times increased incidence of VTE. The risk of VTE is associated with oral but not with transdermal estrogen use.⁵ The Estrogen and ThromboEmbolism Risk (ESTHER) Study documented a 3.5-fold greater risk in women using oral estrogen as compared to a placebo group (95 percent CI 1.8-6.8).3 However, there was no significant difference in VTE rates between the group receiving estrogen transdermally and the group receiving a placebo.³

Oral estrogen is associated with prothrombotic changes in hemostatic factors and an increase in inflammatory markers, such as C-reactive protein, that are seen only minimally with transdermal estrogen. Oral, but not transdermal, estrogen administration is associated with significant reduction in plasma antithrombin III concentrations⁶ and decreased serum tissue-type plasminogen activator (tPA) concentrations.⁷ Raloxifene use is also associated with a significant reduction in plasma antithrombin and tPA levels.⁷ These effects contribute to a procoagulant state and partly explain the increased risk of VTE in both oral estrogen and raloxifene users. These are considered to be consequences of the high hepatic concentration and first-pass effect associated with both orally administered raloxifene and estrogen. When raloxifene is administered orally, approximately 60% of the dose is absorbed, but extensive hepatic conjugation to a number of inactive glucuronides results in an absolute bioavailability of only approximately 2%.

There is therefore a need for developing an effective, transdermal delivery system for raloxifene. Transdermally delivered therapeutic concentrations of raloxifene may avoid, or substantially reduce, high hepatic concentrations, and may reduce or avoid adverse effects on coagulation factors and the consequent risk of VTE, as has been observed with transdermally delivered estrogen. Second, by avoiding extensive first pass metabolism to inactive metabolites, the total amount of raloxifene required to achieve therapeutic concentrations may be reduced, and the adverse effects of metabolites decreased.

### C. Alendronate Background

Alendronate is sparingly water soluble and highly hydrophilic. This is significant, as hydrophilic drugs are difficult to move through skin. Alendronate has a molecular weight of 249.096 g/mol, a melting point of 233-235° C., water solubility of 1 mg/L, a log P of −3.198, and is a solid at room temperature.

Alendronate, sold under the tradename Fosamax™ (oral tablet of alendronate sodium, Merck) is a bisphosphonate drug used for osteoporosis and several other bone diseases. The drug acts as a specific inhibitor of osteoclast-mediated bone resorption. Bisphosphonates are synthetic analogs of pyrophosphate that bind to the hydroxyapatite found in bone. Alendronate sodium is chemically described as (4-amino-1-hydroxybutylidene)bisphosphonic acid monosodium salt trihydrate.

The empirical formula of alendronate sodium is C₄H₁₂NNaO₇P₂.3H₂O and its formula weight is 325.12. The structural formula is:

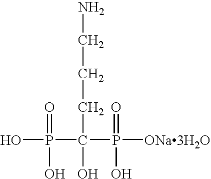

Alendronate sodium is a white, crystalline, nonhygroscopic powder. It is sparingly soluble in water, very slightly soluble in alcohol, and practically insoluble in chloroform.

### D. Naltrexone Background

Naltrexone is water soluble and, like alendronate, is highly hydrophilic. Naltrexone is an opioid receptor antagonist used primarily in the management of alcohol dependence and opioid dependence. It is marketed in generic form as its hydrochloride salt, naltrexone hydrochloride, and marketed under the trade names Revia™ (oral tablet) and Depade™ (oral tablet).

Large doses of naltrexone may cause liver failure. Thus, a dosage form that delivers a controlled amount of drug is highly desirable. Naltrexone is used to help people who have a narcotic or alcohol addiction stay drug free. Naltrexone is used after the patient has stopped taking drugs or alcohol. It works by blocking the effects of narcotics or by decreasing the craving for alcohol. Because subjects taking naltrexone may have had past drug addiction, it is critical to develop a dosage form of naltrexone that deters the subject from taking more than the prescribed drug dosage.

Naltrexone has traditionally been thought of as a drug which is not suitable for transdermal delivery. See e.g., U.S. Pat. No. 4,573,995, which states that ““Although various types of transdermal therapeutic systems for delivering a wide variety of drugs are known to the art . . . [the prior art transdermal references do not teach] the transdermal delivery of either naloxone, naltrexone or nalbuphine . . . [T]he permeability through skin of these drugs is too low to produce any therapeutic effect from a reasonably sized therapeutic system.”

There is a need in the art for transdermal delivery systems that can deliver active agents that cannot be delivered at therapeutic levels using traditional transdermal technology. The present invention satisfies this need.

### SUMMARY

The present invention is directed to transdermal delivery of non-traditional transdermal delivery active agents. Such active agents do not meet the criteria typically required for a successful transdermal delivery formulation to be developed. For example, active agents that can be delivered using traditional transdermal technology have one or more of the following characteristics: (1) a melting point less than about 150° C., (2) a molecular weight less than about 500 Da, (3) a Log P=1 to 3, and/or (4) a reasonably low therapeutically effective dose. The present invention is directed to transdermal delivery dosage forms comprising one or more active agents which have one or more of the following characteristics: (1) a melting point greater than about 150° C., (2) a molecular weight greater than about 500 Da, (3) a Log P that is less than about 1 or greater than about 3, and/or (4) a high therapeutically effective dose. A “high therapeutically effective dose” can be, for example, greater than about 5 mg, greater than about 10 mg, greater than about 13 mg, greater than about 15 mg, greater than about 20 mg, or up to about 25 mg. For example, in one embodiment of the invention, assuming that the BA for transdermal delivery is on the order of about 1%, the maximum drug loading is about 15% (w/w) active agent in the formulation and the maximum amount of drug product applied is on the order of about 10 g, the maximum therapeutic dose is (1%×15%×10 g) about 15 mg. Active agents suitable for transdermal delivery according to the invention are collectively referred to as “non-traditional transdermal active agents.” Examples of such active agents include, but are not limited to, raloxifene, alendronate, and naltrexone.

In one aspect, a composition for transdermal delivery of a non-traditional transdermal active agent, such as raloxifene, alendronate, or naltrexone, is provided, comprising: a non-traditional transdermal active agent, a solvent, a non-miscible liquid, a stabilizer, and water, wherein the composition is formulated into an emulsion and the a non-traditional transdermal active agent is at least partially present in a particulate state. In such embodiments, the delivery of the non-traditional transdermal active agent occurs passively across the skin, with the concentration gradient being the primary delivery mechanism. In some embodiments, the composition is not in a gel dosage form. The solvent may be an alcohol.

In some embodiments, a concentration of about 3 wt % of a non-traditional transdermal active agent such as raloxifene results in a mean cumulative amount of drug diffused of: at least about 0.2 μg/cm² over a period of about 1 hour; at least about 0.3 μg/cm² over a period of about 2 hours; at least about 0.5 μg/cm² over a period of about 4 hours; at least about 0.6 μg/cm² over a period of about 6 hours; at least about 0.7 μg/cm² over a period of about 8 hours; at least about 0.8 μg/cm² over a period of about 12 hours; at least about 0.95 μg/cm² over a period of about 24 hours; or any combination thereof. In some embodiments of the disclosed compositions, a concentration of about 3 wt % of raloxifene results in a flux of about 0.0077 mg/15 cm²/day.

In other embodiments, transdermal compositions are disclosed which exhibit a reduced level of toxicity as compared to an oral dosage form of a non-traditional transdermal active agent, such as raloxifene, alendronate, or naltrexone, comprising the same quantity of the non-traditional transdermal active agent. In some embodiments of the transdermal compositions, the amount of the non-traditional transdermal active agent required to achieve a therapeutic concentration is less than that required for an oral dosage form of the same non-traditional transdermal active agent. For raloxifene, in some such embodiments, the oral dosage may be less than about 160 mg. In other such embodiments, the oral dosage may be less than about 150 mg. In other embodiments, a single transdermal application results in a therapeutic concentration of a non-traditional transdermal active agent such as raloxifene equivalent to or greater than that obtained with a single oral dosage of 60 mg of raloxifene. For alendronate, the oral dosage is typically one 70 mg tablet once weekly. For naltrexone, a dose of 50 mg once daily is recommended for most patients.

In yet other embodiments, the transdermal compositions result in a bioavailability of a non-traditional transdermal active agent, such as raloxifene, alendronate, or naltrexone, of about 1%, correlating with about a 15 cm² area of application. In such embodiments, a single transdermal application comprises from about 1 gram to about 10 grams. For example, in various embodiments, the amount of the single application is about 1 gram, about 2 grams, about 3 grams, about 4 grams, about 5 grams, about 6 grams, about 7 grams, about 8 grams, about 9 grams, or about 10 grams. In other such embodiments, the composition is applied over a cumulative surface area of about 1000 cm² or less, such as less than about 750 cm². While in other embodiments, the compositions comprise up to about 15 wt % of a non-traditional transdermal active agent, such as raloxifene, alendronate, or naltrexone.

In some embodiments of the transdermal compositions, the non-traditional transdermal active agent, such as raloxifene, alendronate, or naltrexone, is at least partially present in a particulate state, and at least partially solubilized in at least one of the solvent, non-miscible liquid, stabilizer, water, or a combination of any two or more thereof. The non-traditional transdermal active agent, such as raloxifene, alendronate, or naltrexone, in the particulate state, may have a diameter of less than about 10 microns.

In other embodiments, the transdermal compositions comprise globules of the non-miscible liquid comprising a dissolved non-traditional transdermal active agent, such as raloxifene, alendronate, or naltrexone, wherein the globules have a diameter of less than about 10 microns.

In another aspect, methods of treating a subject in need of a SERM are provided, comprising applying a raloxifene transdermal composition of the present invention to the skin of the subject. In some embodiments, the transdermal composition is applied as a topical cream or lotion onto the skin of the subject.

In another aspect, methods of treating a subject having a drug or alcohol addiction is provided, comprising applying a naltrexone transdermal composition of the present invention to the skin of the subject. In some embodiments, the transdermal composition is applied as a topical cream or lotion onto the skin of the subject.

In another aspect, methods of treating a subject exhibiting osteoporosis or a similar bone disease is provided, comprising applying an alendronate transdermal composition of the present invention to the skin of the subject. In some embodiments, the transdermal composition is applied as a topical cream or lotion onto the skin of the subject.

Both the foregoing summary and the following brief description of the drawings and the detailed description are exemplary and explanatory and are intended to provide further explanation of the invention as claimed. Other objects, advantages, and novel features will be readily apparent to those skilled in the art from the following detailed description of the invention.

### BRIEF DESCRIPTION OF THE DRAWINGS

FIG. 1 is a graph of mean cumulative amount of drug diffused versus time (μg/cm²).

FIG. 2 depicts the in vivo release profile of a naltrexone hydrochloride transdermal composition according to the invention over time in rabbits

### DETAILED DESCRIPTION

Active agents that can be delivered using traditional transdermal technology have one or more of the following characteristics: (1) a melting point less than about 150° C., (2) a molecular weight less than about 500 Da, (3) a Log P=1 to 3, and/or (4) a reasonably low therapeutically effective dose, i.e., less than about 5 mg. The present invention is directed to transdermal delivery dosage forms comprising one or more active agents which have one or more of the following characteristics: (1) a melting point greater than about 150° C., (2) a molecular weight greater than about 500 Da, (3) a Log P that is less than about 1 or greater than about 3, and/or (4) a high therapeutically effective dose. Active agents suitable for transdermal delivery according to the invention are collectively referred to as “non-traditional transdermal active agents.” Examples of such active agents include, but are not limited to, raloxifene, alendronate, and naltrexone.

Thus, pharmaceutical compositions have been developed to meet the unique needs for affecting the transdermal delivery of non-traditional transdermal active agents, such as raloxifene, alendronate, or naltrexone. Such transdermal compositions comprise at least one non-traditional transdermal active agent, at least partially in a particulate form and at least partially in a solubilized form. U.S. Ser. No. 60/837,294, which is specifically incorporated by reference, discusses methods for preparation of compositions where an API is in both a solid particulate state and in a solubilized state.

Pharmaceutical compositions embodied herein may be formulated in a composition that is an emulsion that resembles a lotion and may be applied to the skin like a lotion. These compositions facilitate the transport of a drug into the superficial layers of the skin where a functional drug depot is created from which the drug continues to diffuse into the systemic circulation. The emulsion deposits the active drug into the stratum corneum and epidermis forming a “patchless patch” on the application area. The drug then gradually diffuses into the deeper layers of the skin until it reaches the bloodstream. Such diffusion results in a stratification of the drug through the various layers of skin, hence the term Stratified Active Pharmaceutical Ingredient Deposition (SAPID). The resulting pharmacokinetic profile is characterized by the absence of major fluctuations in drug serum levels. Advantages of this type of delivery system include the ability of the API to enter the blood-stream quickly, by-pass the gut and the liver, and achieve stable drug levels when applied appropriately. While patch drug delivery shares some of these features, such lotion-like formulations avoid adhesion issues and are cosmetically more acceptable to many patients. In addition, SAPID delivery promises to make transdermal delivery feasible for a larger number of non-traditional transdermal active agents, such as raloxifene, alendronate, or naltrexone, than is currently possible with patch technology, as a more extensive surface area of skin can be used. Beneficially, because the compositions are comprised of oil and water, their products also have moisturizing and conditioning properties. Such compositions also tend to be stable at room temperature, have a high drug payload, a long shelf life, and may be formulated preservative-free.

### A. DEFINITIONS

The present invention is described herein using several definitions, as set forth below and throughout the application.

As used herein, and unless otherwise specified, the term ‘raloxifene’ is used to refer to raloxifene (Formula I), raloxifene hydrochloride (Formula II), other pharmaceutically acceptable salts thereof, or mixtures of any two or more thereof.

As used herein, “about” will be understood by persons of ordinary skill in the art and will vary to some extent depending upon the context in which it is used. If there are uses of the term which are not clear to persons of ordinary skill in the art given the context in which it is used, “about” will mean up to plus or minus 10% of the particular term.

As used herein, the term “non-miscible liquid” refers to a liquid that does not dissolve in another liquid. Non-miscible liquids are capable of forming emulsions.

As used herein, the term “emulsion” refers to a dispersion of one non-miscible liquid in another liquid.

As used herein, the phrase “therapeutically effective amount” shall mean the drug dosage that provides the specific pharmacological response for which the drug is administered in a significant number of subjects in need of such treatment. It is emphasized that a therapeutically effective amount of a drug that is administered to a particular subject in a particular instance will not always be effective in treating the conditions/diseases described herein, even though such dosage is deemed to be a therapeutically effective amount by those of skill in the art.

As used herein, a “non-traditional transdermal active agent” is defined as an active agent having one or more of the following properties: (1) a melting point greater than about 150° C., (2) a molecular weight greater than about 500 Da, (3) a Log P that is less than about 1 or greater than about 3, and/or (4) a high therapeutically effective dose.

A “high therapeutically effective dose” is defined as a dosage greater than about 5 mg, greater than about 6 mg, greater than about 7 mg, greater than about 8 mg, greater than about 9 mg, greater than about 10 mg, greater than about 11 mg, greater than about 12 mg, greater than about 13 mg, greater than about 14 mg, greater than about 15 mg, greater than about 16 mg, greater than about 17 mg, greater than about 18 mg, greater than about 19 mg, greater than about 20 mg, greater than about 21 mg, greater than about 22 mg, greater than about 23 mg, greater than about 24 mg, or up to about 25 mg.

For the purposes of this disclosure and unless otherwise specified, “a” or “an” means “one or more.”

### B. EXEMPLARY EMBODIMENTS OF THE INVENTION

Compositions for transdermal delivery of a non-traditional transdermal active agent, such as raloxifene, alendronate, or naltrexone, may comprise a non-traditional transdermal active agent, a solvent, a non-miscible liquid, a stabilizer, and water. The components may be formulated into an emulsion. Gels are known to be used as topical administration vehicles, however, gels are not an acceptable vehicle for some a non-traditional transdermal active agents, such as raloxifene. As shown below in the Examples section, gel formulations of raloxifene fail to facilitate the flux of raloxifene through skin to an appreciable extent. Thus, in some embodiments, the compositions and formulations of the present invention are not in gel dosage form.

Solvents suitable for use in the embodied compositions include, but are not limited to, isopropyl myristate, triacetin, N-methyl pyrrolidinone, aliphatic and aromatic alcohols, ethanol, dimethyl sulfoxide, dimethyl acetamide, ethoxydiglycol, polyethylene glycols, propylene glycol, or a mixture of any two or more thereof. In other embodiments, the at least one solvent is selected from ethanol, benzyl alcohol, or a combination thereof.

Non-miscible liquids suitable for use in the embodied compositions include, but are not limited to, almond oil (sweet), apricot seed oil, borage oil, canola oil, coconut oil, corn oil, cotton seed oil, fish oil, jojoba bean oil, lard oil, linseed oil (boiled), Macadamia nut oil, medium chain triglycerides, mineral oil, olive oil, peanut oil, safflower oil, sesame oil, soybean oil, squalene, sunflower seed oil, tricaprylin (1,2,3-trioctanoyl glycerol), wheat germ oil, mineral oil (light), or a mixture of any two or more thereof.

Stabilizers suitable for use in the embodied compositions include, but are not limited to, sorbitan esters, glycerol esters, polyethylene glycol esters, block polymers, acrylic polymers, ethoxylated fatty esters, ethoxylated alcohols, ethoxylated fatty acids, monoglycerides, silicon based surfactants, polysorbates, or a mixture of any two or more thereof. The sorbitan ester stabilizer may be selected from Span, Arlacel, or a mixture thereof. The glycerol ester may be glycerin monostearate. The polyethylene glycol ester may be polyethylene glycol stearate. The block polymer may be a PLURONIC™. The acrylic polymer may be PEMULEN™. The ethoxylated fatty ester may be Cremophor RH-40. The ethoxylated alcohol may be BRIJ™. The ethoxylated fatty acid may be TWEEN™ 20.

When a non-traditional transdermal active agent, such as raloxifene, alendronate, or naltrexone, is formulated in the compositions embodied herein, and the transdermal delivery occurs passively across the skin, the primary delivery mechanism is diffusion of the non-traditional transdermal active agent through the skin due to the concentration gradient between the applied formulation and the layers of skin. Without being bound by theory, it is assumed that the solubilized portion of the non-traditional transdermal active agent is readily available for transport through the various layers of skin, while the particulate portion of the non-traditional transdermal active agent, such as raloxifene shown in the examples, is initially blocked by the skin, but is slowly solubilized and later diffuses through the skin as an overall concentration gradient from outer skin to blood stream is established. The combination of solvent, non-miscible liquid, stabilizer, and water determines the extent to which the concentration gradient is established and the flux through the skin over time. The combination of solvent, non-miscible liquid, stabilizer, and water also determines how much non-traditional transdermal active agent, such as raloxifene, alendronate, or naltrexone, is in particulate form and how much is solubilized with optimal formulations providing for the concurrent existence of both solubilized and particulate form of the non-traditional transdermal active agent.

The transdermal compositions may be formulated as lotions and/or creams. In such formulations, and in some embodiments, the compositions provide a concentration of about 3 wt % of a non-traditional transdermal active agent, such as raloxifene, resulting in a mean cumulative amount of drug diffused of at least about 0.2 μg/cm² over a period of about 1 hour. In other embodiments, the mean cumulative amount of non-traditional transdermal active agent such as raloxifene diffused is at least about 0.3 μg/cm² over a period of about 2 hours; at least about 0.5 μg/cm² over a period of about 4 hours, in other embodiments; at least about 0.6 μg/cm² over a period of about 6 hours, in yet other embodiments; at least about 0.7 μg/cm² over a period of about 8 hours, in yet other embodiments; at least about 0.78 g/cm² over a period of about 12 hours, in yet other embodiments; at least about 0.95 μg/cm² over a period of about 24 hours, in yet other embodiments; or in any combination thereof. Such cumulative amounts of non-traditional transdermal active agent, such as raloxifene, diffused may result in a patient receiving a therapeutically effective amount of non-traditional transdermal active agent in the blood stream. In some embodiments, a concentration of about 3 wt % of non-traditional transdermal active agent, such as raloxifene, results in a flux of about 0.0077 mg/15 cm²/day.

Oral dosage forms of non-traditional transdermal active agents, such as raloxifene, alendronate, and naltrexone, are known to give rise to adverse events. For example, oral dosage forms of raloxifene are known to result in a significant reduction in plasma antithrombin and tPA levels, which may contribute to an increased risk of VTE, particularly in combination with oral estrogen dosage forms. These are considered to be consequences of high hepatic concentration and first-pass effects associated with both orally administered raloxifene and estrogen. Side effects of oral administration of alendronate include gastrointestinal irritation, musculoskeletal pain, and headache. In addition, side effects of oral administration of naltrexone include nausea, headache, dizziness, fatigue, insomnia, anxiety, and sleepiness, and with high doses of naltrexone liver failure.

Because transdermal application of non-traditional transdermal active agents, such as raloxifene, alendronate, and naltrexone, has the potential to avoid these effects of oral dosage forms, in some embodiments, the transdermal compositions described herein exhibit a reduced level of toxicity as compared to the oral dosage forms of the same non-traditional transdermal active agent, comprising the same quantity of the non-traditional transdermal active agent.

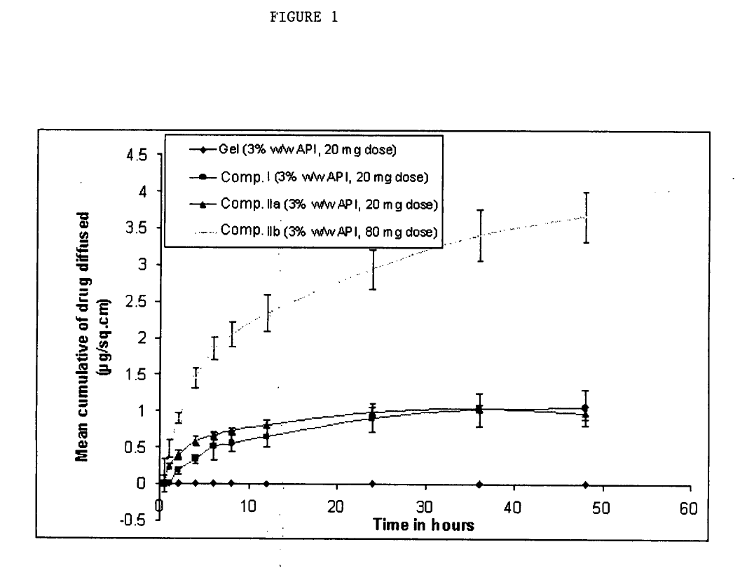

FIG. 1 and Table 1, below, illustrate time dependent mean cumulative amounts of raloxifene diffused through cadaver skin in Franz cells. The results show a direct dose-response relationship between the amount of raloxifene present in the embodied compositions to the skin and the amount of raloxifene diffused through the skin over time. Thus, in some embodiments, the compositions exhibit a direct dose-response relationship such that an increase in the raloxifene dosage correlates with a corresponding increase in flux values. For example, in some embodiments, a two-fold increase in the dosage corresponds to about a two-fold increase in flux values. In other embodiments, a three-fold increase in the dosage corresponds to about a three-fold increase in flux values. In other embodiments, a four-fold increase in the dosage corresponds to about a four-fold increase in flux values. In other embodiments, a five-fold increase in the dosage corresponds to about a five-fold increase in flux values. In other embodiments, a six-fold increase in the dosage corresponds to about a six-fold increase in flux values. In other embodiments, a seven-fold increase in the dosage corresponds to about a seven-fold increase in flux values. In other embodiments, an eight-fold increase in the dosage corresponds to about an eight-fold increase in flux values. In other embodiments, a nine-fold increase in the dosage corresponds to about a nine-fold increase in flux values. In yet other embodiments, a ten-fold increase in the dosage corresponds to about a ten-fold increase in flux values.

In preparing transdermal compositions to achieve certain flux rates or to deliver a given therapeutically effective amount of a non-traditional transdermal active agent, such as raloxifene, alendronate, and naltrexone, to a patient, the amount of the non-traditional transdermal active agent in the composition may vary over a wide range of values. Thus, in some embodiments, the amount of the non-traditional transdermal active agent in the composition ranges from about 0.1 wt % to about 20 wt % of active agent. For example, the transdermal composition may comprise about 0.1 wt %, about 0.5 wt %, about 1 wt %, about 2 wt %, about 3 wt %, about 4 wt %, about 5 wt %, about 6 wt %, about 7 wt %, about 8 wt %, about 9 wt %, about 10 wt %, about 11 wt %, about 12 wt %, about 13 wt %, about 14 wt %, about 15 wt %, about 16 wt %, about 17 wt %, about 18 wt %, about 19 wt %, or about 20 wt % of active agent. In other embodiments, the compositions comprise less than about 5 wt % of active agent. For example, the compositions may have less than about 4.9 wt %, less than about 4.8 wt %, less than about 4.7 wt %, less than about 4.6 wt %, less than about 4.5 wt %, less than about 4.4 wt %, less than about 4.3 wt %, less than about 4.2 wt %, less than about 4.1 wt %, less than about 4.0 wt %, less than about 3.9 wt %, less than about 3.8 wt %, less than about 3.7 wt %, less than about 3.6 wt %, less than about 3.5 wt %, less than about 3.4 wt %, less than about 3.3 wt %, less than about 3.2 wt %, less than about 3.1 wt %, less than about 3.0 wt %, less than about 2.9 wt %, less than about 2.8 wt %, less than about 2.7 wt %, less than about 2.6 wt %, less than about 2.5 wt %, less than about 2.4 wt %, less than about 2.3 wt %, less than about 2.2 wt %, less than about 2.1 wt %, less than about 2.0 wt %, less than about 1.9 wt %, less than about 1.8 wt %, less than about 1.7 wt %, less than about 1.6 wt %, less than about 1.5 wt %, less than about 1.4 wt %, less than about 1.3 wt %, less than about 1.2 wt %, less than about 1.1 wt %, or less than about 1.0 wt % of active agent.

When a non-traditional transdermal active agent, such as raloxifene, is administered orally, approximately 60% of the dose is absorbed but extensive hepatic conjugation to a number of inactive glucuronides results in an absolute bioavailability of 2%. Because of this extreme loss of raloxifene when administered orally, transdermal compositions with lower concentrations of raloxifene, as compared to the oral compositions, may be used to deliver a therapeutically effective amount of raloxifene to the subject. For example, in some embodied transdermal compositions, a bioavailability of about 1% correlates with about a 15 square centimeter (cm²) area of application. When alendronate is administered orally, the systemic bioavailability is low, averaging only 0.6-0.7% in women and in men under fasting conditions. Intake together with meals and beverages other than water further reduces the bioavailability. Finally, when naltrexone is administered orally, naltrexone undergoes rapid and nearly complete absorption with approximately 96% of the dose absorbed from the gastrointestinal tract. However, although well absorbed orally, naltrexone is subject to extensive “first-pass” hepatic metabolism with an oral bioavailability estimate ranging from 5 to 40%.

In other embodiments, the amount of a non-traditional transdermal active agent, such as raloxifene, alendronate, or naltrexone, required to achieve a therapeutically effective amount is less than that required for an oral dosage form of the same non-traditional transdermal active agent. For example, the oral dosage is less than about 150 mg, in some embodiments, or the oral dosage is from about 1 mg to about 150 mg. In other embodiments, the oral dosage is less than about 140 mg. In other embodiments, the oral dosage is less than about 130 mg. In other embodiments, the oral dosage is less than about 120 mg. In other embodiments, the oral dosage is less than about 110 mg. In other embodiments, the oral dosage is less than about 100 mg. In other embodiments, the oral dosage is less than about 90 mg. In other embodiments, the oral dosage is less than about 80 mg. In other embodiments, the oral dosage is less than about 70 mg. In other embodiments, the oral dosage is less than about 60 mg. In other embodiments, the oral dosage is less than about 50 mg. In other embodiments, the oral dosage is less than about 40 mg. In yet other embodiments, the oral dosage is less than about 30 mg, less than about 20 mg, or less than about 10 mg.

A single application of the transdermal compositions may result in a therapeutic concentration of a non-traditional transdermal active agent, such as raloxifene, alendronate, or naltrexone, equivalent to or greater than that obtained with a single oral dosage of the same non-traditional transdermal active agent. In such transdermal compositions, the amount of the single application is from about 1 gram to about 10 grams. For example, in various embodiments, the amount of the single application is about 1 gram, about 2 grams, about 3 grams, about 4 grams, about 5 grams, about 6 grams, about 7 grams, about 8 grams, about 9 grams, or about 10 grams.

As the amount of a non-traditional transdermal active agent, such as raloxifene, alendronate, or naltrexone, in a transdermal composition exhibits a direct dose response relationship such that an increase in the non-traditional transdermal active agent dosage correlates with a corresponding increase in flux values, so too does the amount of non-traditional transdermal active agent delivered increase when the area of composition applied increases. Thus, in some embodiments, the transdermal composition is applied over a cumulative surface area of 1000 cm² or less, such as from about 1 cm² to about 1000 cm². For example, the cumulative surface area of coverage may be 950 cm² or less, 900 cm² or less, 850 cm² or less, 800 cm² or less, 750 cm² or less, 700 cm² or less, 650 cm² or less, 600 cm² or less, 550 cm or less, 500 cm² or less, 450 cm² or less, 400 cm² or less, 350 cm² or less, 300 cm² or less, 250 cm² or less, 200 cm² or less, 150 cm or less, or 100 cm² or less.

In the past, problems existed with transdermal applications for small particulate drugs. Small particles of drug typically provide only small amounts of drug and therefore their usefulness can be limited. In addition, not all drugs can be formulated into small particulate drug dosage forms, as typically such dosage forms are only suitable for poorly water soluble drugs. See e.g., U.S. Pat. No. 5,145,684. However, larger sized particles may have more trouble diffusing across the skin barrier. SAPID systems and compositions were developed to overcome these problems by providing small particles of active agent in an emulsion that readily crosses the skin barrier.

While the sequence of steps may be varied, typically, the non-traditional transdermal active agent, such as raloxifene, alendronate, or naltrexone, is dissolved in a solvent or solvent mixture, followed by addition of a non-miscible liquid, a stabilizer, and water. After homogenization, the resultant emulsion contains the non-traditional transdermal active agent at least partially in a particulate state and at least partially in a solubilized state. Hence, in some embodiments, the transdermal compositions of the present invention comprise a non-traditional transdermal active agent at least partially in a solid particulate state and at least partially in a solubilized state in at least one of the solvent, non-miscible liquid, stabilizer, water, or a mixture of any two or more thereof. For example, in some embodiments, the non-traditional transdermal active agent is present in the transdermal composition at least partially in a particulate state and at least partially in a solubilized state in at least one of the solvent, non-miscible liquid, stabilizer, and water. In other embodiments, the non-traditional transdermal active agent is present in the transdermal composition at least partially in a particulate state and at least partially in a solubilized state in the solvent and the non-miscible liquid. In yet other embodiments, the non-traditional transdermal active agent is present in the transdermal composition at least partially in a particulate state and at least partially in a solubilized state in the solvent, non-miscible liquid, stabilizer, and water.

The amount of non-traditional transdermal active agent, such as raloxifene, alendronate, or naltrexone, in a particulate state and the amount of non-traditional transdermal active agent in a solubilized state has an impact on the effectiveness of the transdermal composition to deliver the non-traditional transdermal active agent. In some embodiments, the amount of non-traditional transdermal active agent in the particulate state ranges from about 5 wt % to about 95 wt %, from about 10 wt % to about 90 wt % in other embodiments, from about 15 wt % to about 85 wt % in other embodiments, from about 20 wt % to about 80 wt % in other embodiments, from about 25 wt % to about 78 wt % in other embodiments, from about 30 wt % to about 75 wt % in other embodiments, from about 35 wt % to about 73 wt % in other embodiments, from about 40 wt % to about 70 wt % in other embodiments, from about 45 wt % to about 70 wt % in other embodiments, from about 50 wt % to about 70 wt % in other embodiments, from about 60 wt % to about 70 wt % in other embodiments, and/or from about 65 wt % to about 70 wt % in yet other embodiments. In some embodiments, the amount of non-traditional transdermal active agent in the solubilized state ranges from about 0.5 wt % to about 80 wt %, from about 1.0 wt % to about 75 wt % in other embodiments, from about 5 wt % to about 70 wt % in other embodiments, from about 10 wt % to about 65 wt % in other embodiments, from about 15 wt % to about 60 wt % in other embodiments, from about 20 to about 55 wt % in other embodiments, from about 25 wt % to about 50 wt % in other embodiments, from about 25 wt % to about 45 wt % in other embodiments, from about 25 wt % to about 40 wt % in other embodiments, from about 28 wt % to about 35 wt % in other embodiments, and/or from about 28 wt % to about 33 wt % in yet other embodiments.

The amount of non-traditional transdermal active agent, such as raloxifene, alendronate, or naltrexone, in a particulate state and the amount of non-traditional transdermal active agent in a solubilized state may also be expressed as a weight ratio of the amount of non-traditional transdermal active agent in a particulate state to the amount of non-traditional transdermal active agent in the solubilized state. For example, such a ratio may range from about 95:5 to about 5:95. In some embodiments, the ratio is about 90:10, about 85:15, about 80:20, about 75:25, about 70:30, about 65:35, about 60:40, about 55:45, about 50:50, about 45:55, about 40:60, about 35:65, about 30:70, about 25:75, about 20:80, about 15:85, about 10:90, or about 5:95.

The size of the particulates of the non-traditional transdermal active agent, such as raloxifene, alendronate, or naltrexone, may also have an impact on diffusion through the skin. In some embodiments, the diameter of the non-traditional transdermal active agent particles in the transdermal compositions is less than about 10 microns. For example, the diameter of the non-traditional transdermal active agent particles may be less than about 9 microns, less than about 8 microns, less than about 7 microns, less than about 6 microns, less than about 5 microns, less than about 4 microns, less than about 3 microns, less than about 2 microns, or about 1 micron or greater. In other embodiments, the diameter of the non-traditional transdermal active agent particles is less than about 1 micron, such as from about 1 nm to about 1 micron. For example, the diameter of the non-traditional transdermal active agent particles may be less than about 900 nm, less than about 800 nm, less than about 700 nm, less than about 600 nm, less than about 500 nm, less than about 400 nm, less than about 300 nm, less than about 290 nm, less than about 280 nm, less than about 270 nm, less than about 260 nm, less than about 250 nm, less than about 240 nm, less than about 230 nm, less than about 220 nm, less than about 210 nm, less than about 200 nm, less than about 190 nm, less than about 180 nm, less than about 170 nm, less than about 160 nm, less than about 150 nm, less than about 140 nm, less than about 130 nm, less than about 120 nm, less than about 110 nm, less than about 100 nm, less than about 90 nm, less than about 80 nm, less than about 70 nm, less than about 60 nm, less than about 50 nm, less than about 40 nm, less than about 30 nm, less than about 20 nm, or less than about 10 nm.

Transdermal compositions embodied herein may be formulated as emulsions. Thus, in some embodiments, the emulsions comprise globules of a non-miscible liquid comprising a dissolved non-traditional transdermal active agent, such as raloxifene, alendronate, or naltrexone. Just as the size of the individual particles of non-traditional transdermal active agent may have a bearing on the diffusion of non-traditional transdermal active agent through the skin, so too may the size of the globules. Therefore, also provided are embodiments where the globules of dissolved non-traditional transdermal active agent have a diameter of less than about 10 microns, such as from about 1 nm to about 10 microns. For example, the globule may have a diameter of less than about 10 microns, less than about 9 microns, less than about 8 microns, less than about 7 microns, less than about 6 microns, less than about 5 microns, less than about 4 microns, less than about 3 microns, less than about 2 microns, less than about 1000 nm, less than about 900 nm, less than about 800 nm, less than about 700 nm, less than about 600 nm, less than about 500 mm, less than about 400 nm, less than about 300 mm, less than about 290 nm, less than about 280 mm, less than about 270 nm, less than about 260 nm, less than about 250 nm, less than about 240 nm, less than about 230 nm, less than about 220 nm, less than about 210 nm, less than about 200 nm, less than about 190 nm, less than about 180 nm, less than about 170 nm, less than about 160 nm, less than about 150 nm, less than about 140 nm, less than about 130 nm, less than about 120 nm, less than about 110 nm n, less than about 100 nm, less than about 90 nm, less than about 80 nm, less than about 70 nm, less than about 60 nm, less than about 50 nm, less than about 40 nm, less than about 30 nm, less than about 20 mm, or less than about 10 nm.

Methods of using the embodied transdermal compositions are also provided. As discussed above, the compositions may be applied directly to the skin as a lotion or cream. The compositions may be applied to any area of skin including, but not limited to, the legs, including anterior and posterior portions of the thigh, calf, and shin; the buttocks; the torso, including the abdomen, chest, breasts, back, and arms; and the scalp. The region of skin is unlimited by the present disclosure. Physicians may want targeted areas of application, or the subject may want the area of application to be easily accessible and/or easily hidden by clothing or medical dressings. Thus, methods are provided comprising applying the transdermal composition of embodied above to the skin of the subject. In other embodiments, the transdermal composition is applied as a topical cream or lotion onto the skin of the subject.

One skilled in the art will readily realize that all ranges and ratios discussed can and do necessarily also describe all subranges and subratios therein for all purposes and that all such subranges and subratios also form part and parcel of this invention. Any listed range or ratio can be easily recognized as sufficiently describing and enabling the same range or ratio being broken down into at least equal halves, thirds, quarters, fifths, tenths, etc. As a non-limiting example, each range or ratio discussed herein can be readily broken down into a lower third, middle third and upper third, etc.

All publications, patent applications, issued patents, and other documents referred to in this specification are herein incorporated by reference as if each individual publication, patent application, issued patent, or other document was specifically and individually indicated to be incorporated by reference in its entirety. Definitions that are contained in text incorporated by reference are excluded to the extent that they contradict definitions in this disclosure.

The present invention, thus generally described, will be understood more readily by reference to the following examples, which are provided by way of illustration and are not intended to be limiting of the present invention.

### EXAMPLES

The following data and descriptions are provided to show results obtained using various models for testing of raloxifene transdermal compositions. The methods of example 1 are applicable to other non-traditional transdermal active agents, such as alendronate and naltrexone.

### Example 1

### A. In Vitro Evaluation of Raloxifene Formulations

Raloxifene formulations were prepared for in vitro testing to evaluate the flux of the drug through the skin of a patient as determined using Franz cells. Raloxifene base (3.0 wt %) was dissolved in a mixture of ethanol (7.0 wt %) and benzyl alcohol (13.0 wt %) at 60° C., with continuous mixing, over 15 minutes. After cooling to room temperature, soybean oil (40.0 wt %) was added with continued mixing, followed by the addition of Poloxamer 188 (5.0 wt %) dissolved in water (32.0 wt %). The resultant mixture was then put into a homogenizer (APV-1000) and passed at high pressure three times (10,000 psi) to obtain the final formulation. As prepared by this procedure, the raloxifene in the final formulation is present in both particulate (approximately 70%) and solubilized (approximately 30%) states.

Franz cells were used to evaluate the flux of raloxifene, in various formulations, through cadaver skin. The formulations tested were a gel formulation (3 wt % raloxifene, 20 mg dose), 2 raloxifene compositions (3 wt % raloxifene, 20 mg dose), and a third raloxifene composition (3 wt % raloxifene, 80 mg dose). The gel formulation was used as the control, with 3 of 4 Franz cells showing no flux and the remaining one showing minimal flux. Therefore, for each time at which the flux was recorded, the gel formulations were given a value of zero flux and all other formulations were then normalized to this zero point. The results are presented in Table 1, below, and graphically in FIG. 1.

TABLE 1
Flux of Raloxifene as Measured Using Franz Cells

| Formulation | Flux (mg/15 cm²/day) |
|---|---|
| Gel (3 wt % API, 20 mg dose) | 0 |
| Composition I (3 wt % API, 20 mg dose) | 0.0077 |
| Composition IIa (3 wt % API, 20 mg dose) | 0.0059 |
| Composition IIb (3 wt % API, 80 mg dose) | 0.0238 |

As is evident from the data in Table 1, there is a direct dose-response relationship in which a 4-fold increase in dose results in an approximately 4-fold increase in flux. The flux values presented in Table 1 and in FIG. 1 may result in a bioavailability of raloxifene of about 1% over a 15 cm² area. Extrapolating these results, a 4 gram application of 3 wt % raloxifene over two thighs of a subject (2×375 cm²) may be equivalent to a 60 mg oral tablet, thus providing an exemplary clinically acceptable formulation.

### B. Toxicology

### Carcinogenesis⁸

In a 21-month carcinogenicity study in mice, there was an increased incidence of ovarian tumors in female animals given 9 to 242 mg/kg, which included benign and malignant tumors of granulosa/theca cell origin and benign tumors of epithelial cell origin. Systemic exposure (AUC) of raloxifene in this group was 0.3 to 34 times that in postmenopausal women administered a 60-mg dose. There was also an increased incidence of testicular interstitial cell tumors, prostatic adenomas, and adenocarcinomas in male mice given 41 or 210 mg/kg (4.7 or 24 times the AUC in humans), and prostatic leiomyoblastoma in male mice given 210 mg/kg.

In a 2-year carcinogenicity study in rats, an increased incidence in ovarian tumors of granulosa/theca cell origin was observed in female rats given 279 mg/kg (approximately 400 times the AUC in humans). The female rodents in these studies were treated during their reproductive lives when their ovaries were functional and responsive to hormonal stimulation.

### C. Toxicology

### Mutagenesis⁸

Raloxifene.HCl was not genotoxic in any of the following test systems: (a) the Ames test for bacterial mutagenesis with and without metabolic activation, (b) the unscheduled DNA synthesis assay in rat hepatocytes, (c) the mouse lymphoma assay for mammalian cell mutation, (d) the chromosomal aberration assay in Chinese hamster ovary cells, (e) the in vivo sister chromatid exchange assay in Chinese hamsters, and (f) the in vivo micronucleus test in mice.

### D. Dermal Tolerance

The potential to produce dermal irritation was evaluated in a repeat application study in guinea pigs using raloxifene transdermal formulations embodied herein and placebo formulations containing no raloxifene. Four animals per test article were prepared by clipping their sides and back free of hair the day prior to the first application. Test articles (250 mg of each) were applied to the same 12.5 cm² site on each of the animals daily, for 14 days. On each application, the test article was applied directly to the skin and rubbed in until no longer visible. Four adjacent areas of skin on each animal served as controls. Sites of application were observed for erythema and edema, and scored using the Draize Scale. Animal weights were monitored pretreatment and weekly thereafter. Physical signs were evaluated daily.

Daily group average erythema and edema scores were derived for each time period. The daily group average erythema score was calculated by adding the individual erythema scores for each time period and dividing by the number of scores. The procedure was repeated using the individual edema scores to derive the daily group average edema score.

In the group receiving the raloxifene formulation, the daily group average irritation score was 0. Individual erythema scores were 0, and there was no edema noted at any observation period. There were no abnormal physical signs noted. Body weight changes were normal.

In the group receiving the placebo formulation, the daily group average irritation scores ranged from 0 to 0.125. Individual erythema scores ranged from 0 to 1, and there was no edema noted at any observation period. There were no abnormal physical signs noted. Body weight changes were normal.

### E. Pharmacokinetics⁸

The disposition of orally administered raloxifene has been evaluated in more than 3000 postmenopausal women in selected raloxifene osteoporosis treatment and prevention clinical trials using a population approach. Pharmacokinetic data were also obtained in conventional pharmacology studies in 292 postmenopausal women. Raloxifene exhibits high within-subject variability (approximately 30% coefficient of variation) of most pharmacokinetic parameters. Table 2 summarizes the pharmacokinetic parameters of raloxifene.

TABLE 2
Summary of Raloxifene Pharmacokinetic Parameters
in the Healthy Postmenopausal Woman.

| | Cₘₐₓ^{a} | | AUC_{∞}^{a} | | |
|---|---|---|---|---|---|
| | (ng/mL)/ | | (ng · hr/mL)/ | CL/F | V/F |
| | (mg/kg) | t_{1/2} (hr) | (mg/kg) | (L/kg · hr) | (L/kg) |
| Single | | | | | |
| Dose | | | | | |
| Mean | 0.50 | 27.7 | 27.2 | 44.1 | 2348 |
| CV (%) | 52 | 10.7 to 273^{b} | 44 | 46 | 52 |
| Multiple | | | | | |
| Dose | | | | | |
| Mean | 1.36 | 32.5 | 24.2 | 47.4 | 2583 |
| CV (%) | 37 | 15.8 to 86.6^{b} | 36 | 41 | 56 |

Abbreviations:
Cₘₐₓ = maximum plasma concentration,
t_{1/2} = half-life,
AUC = area under the curve,
CL = clearance,
V = volume of distribution,
F = bioavailability,
CV = coefficient of variation.
^{a}Data normalized for dose in mg and body weight in kg.
^{b}Range of observed half-life.

### F. Metabolism⁸

Biotransformation and disposition of raloxifene in humans have been determined following oral administration of ¹⁴C-labeled raloxifene. Raloxifene undergoes extensive first-pass metabolism to the glucuronide conjugates: raloxifene-4′-glucuronide, raloxifene-6-glucuronide, and raloxifene-6,4′-diglucuronide. No other metabolites have been detected, providing strong evidence that raloxifene is not metabolized by cytochrome P450 pathways. Unconjugated raloxifene comprises less than 1% of the total radiolabeled material in plasma. The terminal log-linear portions of the plasma concentration curves for raloxifene and the glucuronides are generally parallel. This is consistent with interconversion of raloxifene and the glucuronides metabolites. Following intravenous administration, raloxifene is cleared at a rate approximating hepatic blood flow. Apparent oral clearance is 44.1 L/kg·hr. Raloxifene and its glucuronide conjugates are interconverted by reversible systemic metabolism and enterohepatic cycling, thereby prolonging its plasma elimination half-life to 27.7 hours after oral dosing. Results from single oral doses of raloxifene predict multiple-dose pharmacokinetics. Following chronic dosing, clearance ranges from 40 to 60 L/kg.hr. Increasing doses of raloxifene HCl (ranging from 30 to 150 mg) result in slightly less than a proportional increase in the area under the plasma time concentration curve (AUC).

### G. Excretion⁸

Raloxifene is primarily excreted in feces, and less than 0.2% is excreted unchanged in urine. Less than 6% of the raloxifene dose is eliminated in urine as glucuronide conjugates.

### H. Special Populations⁸

No differences in raloxifene pharmacokinetics were detected with regard to age (range 42 to 84 years). Total extent of exposure and oral clearance, normalized for lean body weight, are not significantly different between age-matched female and male volunteers. There were no discernible differences in raloxifene plasma concentrations among racial groups.

### Example 2

Naltrexone

Ethanol, soybean oil and polysorbate 80 were mixed together (Table 3). Naltrexone HCl was then dissolved in water and added to the solvent/oil/stabilizer mixture under high-shear mixing (Silverson high-speed mixer) at 9000 rpm. The homogenizer was run for about 3 minutes to obtain an emulsion. The pH of the resulting composition was then adjusted with citric acid to a pH of 6.76.

TABLE 3

| Ingredient | Quantity |
|---|---|
| Naltrexone HCl | 0.2 gm |
| Ethanol | 8.8 gm |
| Polysorbate 80 | 9.4 gm |
| Soybean oil | 50.2 gm |
| Water | 31.4 gm |
| Citric acid | q.s to pH 6.7 |

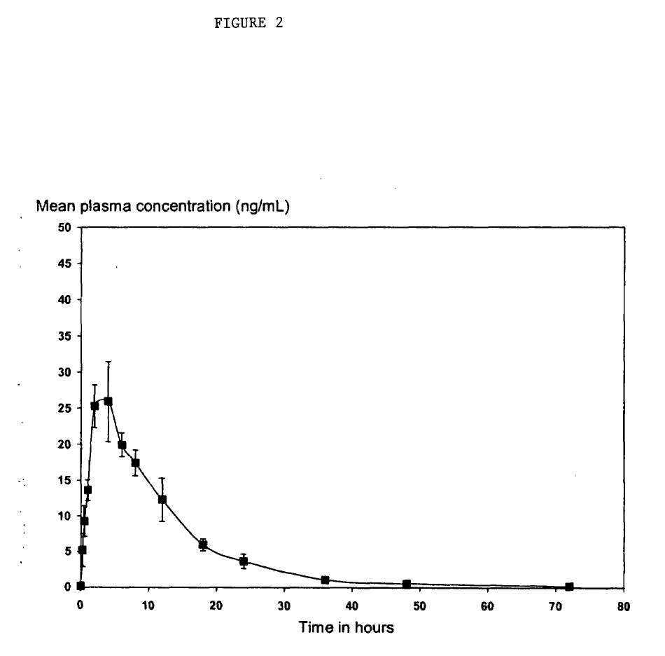

FIG. 2 depicts the in vivo release profile of naltrexone hydrochloride from the formulation shown in Table 3 over time in rabbits. The formulation (2 ml of the formulation containing 10 mg of Naltrexone HCl per gram formulation) was applied topically to three male rabbits. Following administration, blood samples were taken from the rabbits at time 0 and at periodic intervals following administration: 0 (pre-dose), 0.5, 1, 2, 4, 6, 8, 12, 18, 24, 36, 48 hours post-dose. Serum levels of naltrexone hydrochloride were then measured in each blood sample as determined by liquid chromatography-mass spectrometry (LC-MS).

While some embodiments have been illustrated and described, it should be understood that changes and modifications can be made therein in accordance with ordinary skill in the art without departing from the invention in its broader aspects as defined in the following claims.

### REFERENCES

1. McKinnie, J., Drug Delivery Technology 6(8), 54-58 (2006).
2. Scarabin P. Y., Alhenc-Gelas M., Plu-Bureau G., Taisne P., Agher R., Aiach M. Arterioscler Thromb Vase Biol. 17(11) 3071-8 (1997).
3. Scarabin, P. Y., Oger, E., Plu-Bureau, G. Lancet 362(9382) 428-32 (2003).
4. Ozeren, M., Karahan, S. C., Ozgur, M., Eminagaoglu, S., Unsal, M., Baytan, S., Bozkaya, H. Acta Obstetricia et Gynecologica Scandinavica 84, 987 (2005)
5. Mueck, A. O., Seeger, H., Lippert, T. H. Gynakol Geburtshilfliche Rundsch 40(2) 61-7 (2000).
6. Bonduki, C. E., Lourenco, D. M., Baracat, E., Haidar, M., Noguti, M. A., da Motta, E. L., Lima, G. R. Acta Obstet Gynecol Scand. 77(3) 330-3 (1998).
7. Giltay, E. J., Gooren, L. J. G., Emeis, J. J., Kooistra, T., Stehouwe, C. D. A. Arteriosclerosis, Thrombosis, and Vascular Biology 20, 1396 (2000).
8. U.S. prescribing information (dated 11 Jul., 2003) for Evista® (Raloxifene.HCl) Tablets. Eli Lilly and Company, Indianapolis, Ind. 46285, Lilly document PV 3086 AMP.

What is claimed is:

1. A composition for transdermal delivery of an active agent comprising:
(a) at least one active agent, wherein the active agent has one or more of the following properties: (i) a melting point greater than about 150° C., (ii) a molecular weight greater than about 500 Da, (iii) a Log P that is less than about 1 or greater than about 3, and (4) a high therapeutically effective dose;
(b) a solvent;
(c) a non-miscible liquid;
(d) a stabilizer; and
(e) water;
wherein the composition is formulated into an emulsion and the raloxifene is at least partially present in a particulate state.

2. The composition of claim 1, wherein the active agent is selected from the group consisting of raloxifene, alendronate, and naltrexone.

3. The composition of claim 1, wherein delivery of the active agent occurs passively across the skin, with the concentration gradient being the primary delivery mechanism.

4. The composition of claim 1, wherein a concentration of about 3 wt % of active agent results in a mean cumulative amount of drug diffused of:
(a) at least about 0.2 μg/cm² over a period of about 1 hour;
(b) at least about 0.3 μg/cm² over a period of about 2 hours;
(c) at least about 0.5 μg/cm² over a period of about 4 hours;
(d) at least about 0.6 μg/cm² over a period of about 6 hours;
(e) at least about 0.7 μg/cm² over a period of about 8 hours;
(f) at least about 0.8 μg/cm² over a period of about 12 hours;
(g) at least about 0.95 μg/cm² over a period of about 24 hours; or
(h) any combination thereof.

5. The composition of claim 1 which exhibits a reduced level of toxicity as compared to an oral dosage form of the same active agent, comprising the same quantity of active agent.

6. The composition of claim 1, wherein the composition exhibits a direct dose response relationship such that an increase in the active agent dosage correlates with a corresponding increase in flux values.

7. The composition of claim 1, wherein an amount of active agent required to achieve a therapeutic concentration is less than that required for an oral dosage form of the same active agent.

8. The composition of claim 7, wherein the oral dosage is less than about 150 mg, less than about 140 mg, less than about 130 mg, less than about 120 mg, less than about 110 mg, less than about 100 mg, less than about 90 mg, less than about 80 mg, less than about 70 mg, less than about 60 mg, less than about 50 mg, less than about 40 mg, less than about 30 mg, less than about 20 mg, or less than about 10 mg.

9. The composition of claim 1, wherein a single application results in a therapeutic concentration of active agent equivalent to or greater than that obtained with a single oral dosage of the same active agent.

10. The composition of claim 9, wherein the single application comprises about 1 gram, about 2 grams, about 3 grams, about 4 grams, about 5 grams, about 6 grams, about 7 grams, about 8 grams, about 9 grams, or about 10 grams of the composition of claim 1.

11. The composition of claim 1, wherein the composition is applied over a cumulative surface area of 1000 cm² or less, 950 cm² or less, 900 cm² or less, 850 cm² or less, 800 cm² or less, 750 cm² or less, 700 cm² or less, 650 cm² or less, 600 cm² or less, 550 cm² or less, 500 cm or less, 450 cm² or less, 400 cm² or less, 350 cm² or less, 300 cm² or less, 250 cm² or less, 200 cm² or less, 150 cm² or less, or 100 cm² or less.

12. The composition of claim 1, comprising about 0.1 wt %, about 0.5 wt %, about 1 wt %, about 2 wt %, about 3 wt %, about 4 wt %, about 5 wt %, about 6 wt %, about 7 wt %, about 8 wt %, about 9 wt %, about 10 wt %, about 11 wt %, about 12 wt %, about 13 wt %, about 14 wt %, or about 15 wt % of active agent.

13. The composition of claim 1, comprising less than about 5.0 wt %, less than about 4.9 wt %, less than about 4.8 wt %, less than about 4.7 wt %, less than about 4.6 wt %, less than about 4.5 wt %, less than about 4.4 wt %, less than about 4.3 wt %, less than about 4.2 wt %, less than about 4.1 wt %, less than about 4.0 wt %, less than about 3.9 wt %, less than about 3.8 wt %, less than about 3.7 wt %, less than about 3.6 wt %, less than about 3.5 wt %, less than about 3.4 wt %, less than about 3.3 wt %, less than about 3.2 wt %, less than about 3.1 wt %, less than about 3.0 wt %, less than about 2.9 wt %, less than about 2.8 wt %, less than about 2.7 wt %, less than about 2.6 wt %, less than about 2.5 wt %, less than about 2.4 wt %, less than about 2.3 wt %, less than about 2.2 wt %, less than about 2.1 wt %, less than about 2.0 wt %, less than about 1.9 wt %, less than about 1.8 wt %, less than about 1.7 wt %, less than about 1.6 wt %, less than about 1.5 wt %, less than about 1.4 wt %, less than about 1.3 wt %, less than about 1.2 wt %, less than about 1.1 wt %, or less than about 1.0 wt % of active agent.

14. The composition of claim 1, wherein the active agent:
(a) is at least partially solubilized in at least one of the solvent, non-miscible liquid, stabilizer and water;
(b) is at least partially present in a particulate state and is at least partially solubilized in at least one of the solvent, non-miscible liquid, stabilizer, water, or a combination of any two or more thereof;
(c) is at least partially present in a particulate state and is at least partially solubilized in the solvent and the non-miscible liquid; or
(d) is at least partially present in a particulate state and is at least partially solubilized in the solvent, non-miscible liquid, stabilizer, and water.

15. The composition of claim 14, wherein the diameter of the active agent particles is less than about 10 microns.

16. The composition of claim 15, wherein the diameter of the active agent particles is less than about 9 microns, less than about 8 microns, less than about 7 microns, less than about 6 microns, less than about 5 microns, less than about 4 microns, less than about 3 microns, less than about 2 microns, about 1 micron or greater, less than about 1 micron, less than about 900 nm, less than about 800 nm, less than about 700 nm, less than about 600 nm, less than about 500 nm, less than about 400 nm, less than about 300 nm, less than about 290 nm, less than about 280 nm, less than about 270 nm, less than about 260 nm, less than about 250 nm, less than about 240 nm, less than about 230 nm, less than about 220 nm, less than about 210 nm, less than about 200 nm, less than about 190 nm, less than about 180 nm, less than about 170 nm, less than about 160 nm, less than about 150 nm, less than about 140 nm, less than about 130 nm, less than about 120 nm, less than about 110 nm, less than about 100 nm, less than about 90 nm, less than about 80 nm, less than about 70 nm, less than about 60 nm, less than about 50 nm, less than about 40 nm, less than about 30 nm, less than about 20 nm, or less than about 10 nm.

17. The composition of claim 1, further comprising globules of the non-miscible liquid comprising dissolved active agent, wherein the globules have a diameter selected from the group consisting of less than about 10 microns, less than about 9 microns, less than about 8 microns, less than about 7 microns, less than about 6 microns, less than about 5 microns, less than about 4 microns, less than about 3 microns, less than about 2 microns, less than about 1000 nm, less than about 900 nm, less than about 800 nm, less than about 700 nm, less than about 600 nm, less than about 500 nm, less than about 400 nm, less than about 300 nm, less than about 290 nm, less than about 280 nm, less than about 270 nm, less than about 260 nm, less than about 250 nm, less than about 240 nm, less than about 230 nm, less than about 220 nm, less than about 210 nm, less than about 200 nm, less than about 190 nm, less than about 180 nm, less than about 170 nm, less than about 160 nm, less than about 150 nm, less than about 140 nm, less than about 130 nm, less than about 120 nm, less than about 110 nm, less than about 100 nm, less than about 90 nm, less than about 80 nm, less than about 70 nm, less than about 60 nm, less than about 50 nm, less than about 40 nm, less than about 30 nm, less than about 20 nm, or less than about 10 nm.

18. The composition of claim 1, wherein the non-miscible liquid is selected from almond oil, apricot seed oil, borage oil, canola oil, coconut oil, corn oil, cotton seed oil, fish oil, jojoba bean oil, lard oil, linseed oil, Macadamia nut oil, medium chain triglycerides, mineral oil, olive oil, peanut oil, safflower oil, sesame oil, soybean oil, squalene, sunflower seed oil, 1,2,3-trioctanoyl glycerol, wheat germ oil, mineral oil, or a mixture of any two or more thereof.

19. The composition of claim 1, wherein the solvent is selected from isopropyl myristate, triacetin, N-methyl pyrrolidinone, aliphatic and aromatic alcohols, ethanol, dimethyl sulfoxide, dimethyl acetamide, ethoxydiglycol, polyethylene glycols, propylene glycol, or a mixture of any two or more thereof.

20. The composition of claim 1, wherein the solvent is an alcohol.

21. The composition of claim 20, wherein the solvent is ethanol, benzyl alcohol, or a combination thereof.

22. The composition of claim 1, wherein the stabilizer is selected from sorbitan esters, glycerol esters, polyethylene glycol esters, block polymers, acrylic polymers, ethoxylated fatty esters, ethoxylated alcohols, ethoxylated fatty acids, monoglycerides, silicon based surfactants, polysorbates, or a mixture of any two or more thereof.

23. The composition of claim 22, wherein the sorbitan ester stabilizer is Span, Arlacel, or a mixture thereof, wherein the glycerol ester is glycerin monostearate, wherein the polyethylene glycol ester is polyethylene glycol stearate, wherein the block polymer is a PLURONIC™, wherein the acrylic polymer is PEMULEN™, wherein the ethoxylated fatty ester is Cremophor RH-40, wherein the ethoxylated alcohol is BRIJ™, and wherein the ethoxylated fatty acid is TWEEN™ 20.

24. A method of treating a subject in need of a selective estrogen receptor modulator comprising applying the composition of claim 1 to the skin of the subject.

25. The method of claim 24, wherein the composition is applied as a topical cream onto the skin of the subject.
